(19) United States
(12) Patent Application Publication
(10) Pub. No.: US 2010/0227324 A1
(43) Pub. Date: Sep. 9, 2010

(54) MEASUREMENT OF ARYLESTERASE ENZYMATIC ACTIVITY AND ASSESSMENT OF GENETIC POLYMORPHISMS LOCATED IN THE PON1 GENE AS A DIAGNOSTIC TOOL IN AUTISM-SPECTRUM DISORDERS

(76) Inventors: Antonio Persico, Rome (IT); Roberto Sacco, Rome (IT); Carla Lintas, Rome (IT)

Correspondence Address: OCCHIUTI ROHLICEK & TSAO, LLP, 10 FAWCETT STREET, CAMBRIDGE, MA, 02138, US

(21) Appl. No.: 12/681,179

(22) PCT Filed: Oct. 10, 2008
(86) PCT No.: PCT/EP08/63612

§ 371 Date: Apr. 1, 2010

Related U.S. Application Data
(60) Provisional application No. 60/979,423, filed on Oct. 12, 2007.

(51) Int. Cl. C12Q 1/68; C12Q 1/44

(52) U.S. Cl. 435/6; 435/19

(57) ABSTRACT

The present invention concerns a method for detecting the presence of or predisposition to autism, an autism spectrum disorder, or an autism-associated disorder in a subject, the method comprising measuring an arylesterase enzymatic activity in a sample from the subject, optionally combined with the determination of alleles of PON1 polymorphisms.

### FIELD OF THE INVENTION

The present invention relates generally to the fields of genetics and medicine.

### BACKGROUND OF THE INVENTION

Autism is a severe Pervasive Developmental Disorder characterized by impaired language, communication and social skills, as well as by repetitive and stereotypic patterns of behavior. The onset of autism occurs by definition before 3 years of age; it is usually insidious, sometimes more abrupt, in other instances characterized by the loss of previously-acquired social functions (“regression”). Its incidence has dramatically risen from 2-5 to 15-60/10,000 children during the last two decades: broader diagnostic criteria and increased medical awareness have contributed to determine this trend, but the involvement of environmental factors is also likely. Despite strong familial components, clinical and genetic complexities have posed a major challenge to our understanding of this disease. Clinically, the behavioral expression of autism-predisposing genes can range from minimal personality traits to full-blown autism, identifying a broad clinical entity referred to as “autism-spectrum disorder” (ASD), which includes autistic disorder (Kanner's “autism”), childhood disintegrative disorder, pervasive development disorder not otherwise specified (PDD-NOS or “atypical autism”) and Asperger syndrome. Genetically, the picture is complicated by significant interindividual heterogeneity, numerous contributing loci, incomplete penetrance (i.e., unaffected individuals carrying autism-vulnerability genes), phenocopies (i.e., autistic individuals carrying no genetic predisposition and affected only due to environmental factors), gene-gene and gene-environment interactions.³

Several lines of evidence strongly support a prenatal onset for developmental abnormalities later leading to autism: post-mortem assessments of brains of autistic patients have unveiled early neurodevelopmental alterations including reduced programmed cell death and/or increased cell proliferation, altered cell migration, abnormal cell differentiation with reduced neuronal size, and altered synaptogenesis; many children later diagnosed with ASD display motor abnormalities and/or excessive body growth, already on the day of birth or in early neonatal life. In addition, systemic signs and symptoms including macrosomy, non-specific enterocolitis, immune dysreactivity and renal oligopeptiduria, pose autism as a multi-organ systemic disorder encompassing several developmental components, not restricted to the central nervous system.

Approximately 10% of ASD patients suffer from syndromic autism. In these patients, autism is one of several symptoms part of a broader syndrome of known origin. Syndromes accompanied by autism include several genetic diseases (fragile-X, tuberous sclerosis, neurofibromatosis, Angelman, trisomy 21, Cornelia de Lange, Smith-Lemli-Opitz, de novo chromosomal rearrangements) and rare teratological conditions, such as prenatal exposure to drugs like thalidomide and valproic acid. Currently-available genetic, biochemical, and genomic techniques, coupled with an accurate family history, allow the precise identification of these syndromes.

The remaining 90% of ASD patients suffers from “non-syndromic” autism, meaning that all genetic, biochemical and genomic exams have excluded known causes of syndromic autism, yet the patient appears autistic upon behavioral observation. One of the most dramatic consequences of our limited understanding of autism pathophysiology is that until now all attempts to identify a biochemical, genetic, or radiological test able to diagnose autism per se in non-syndromic cases have failed. In the absence of any laboratory testing, to this date autism is diagnosed exclusively on the basis of behavioral observation. Despite the efforts of expert clinicians, this limitation obviously leads to delayed and sometime mistaken diagnoses, with delays in applying appropriate therapies and rehabilitation programs.

Therefore, a biochemical, genetic or radiological test able to at least partly distinguish between individuals suffering from autism-spectrum disorders and normal subjects would represent an immense contribution to clinical practice, by:
1) Corroborating and reinforcing diagnoses initially based on behavioral observation, in suspect cases of autism-spectrum disorder;
2) Redirecting the diagnostic process in the presence of atypical symptoms or when the clinical diagnosis is uncertain (this occurs most frequently in the presence of Pervasive Developmental Disorders Not Otherwise Specified);
3) Allowing earlier diagnoses, conceivably even on the day of birth. This would foster early prevention programs to be implemented in neonates and children well before age 3;
4) Possibly providing parameters useful also to follow-up on the efficacy of treatment interventions;
5) Identifying normal individuals carrying autism-vulnerability genes and at risk for having autistic children. The identification of healthy carriers would in turn pave the path to preventive interventions aimed at correcting the developmental deficits leading to autism still during pregnancy.

### SUMMARY OF THE INVENTION

The present invention provides a test that, by combining biochemical and genetic information, for the first time distinguishes reliably most autistic patients from normal individuals. This test also allows the identification of normal individuals predisposed to having autistic children. The test is based upon the parallel assessment of (a) one biochemical parameter, namely arylesterase enzymatic activity measured in serum following blood drawing, and (b) two genetic polymorphisms located in the paraoxonase (PON1) gene, namely the Q192R and C-108T single nucleotide polymorphisms (SNPs). The combination of information regarding these two genetic variants located in the promoter (C-108T) and coding sequence (Q192R) of the PON1 gene, and arylesterase activity exerted in serum by the protein product of PON1 gene expression, represents a diagnostic tool able to provide clinicians with extremely useful diagnostic information.

Accordingly, the present invention concerns a method for detecting the presence of or predisposition to autism, an autism spectrum disorder, or an autism-associated disorder in a subject, the method comprising measuring an arylesterase enzymatic activity in a biological sample of said subject, a low arylesterase enzymatic activity being indicative of the presence of or the predisposition to autism, an autism spectrum disorder, or an autism-associated disorder. Preferably, the method further comprises detecting the presence of an alteration in the PON1 gene locus in a biological sample of said subject. The alteration can be one or several SNP(s). Preferably, said SNP is C-108T and/or Q192R. In particular, the presence of an allele T of SNP C-108T and/or of an allele R of SNP Q192R in a subject is indicative of the presence of or the predisposition to autism, an autism spectrum disorder, or an autism-associated disorder. Preferably, the biological sample is a blood sample, preferably a serum sample. Then, a low arylesterase enzymatic activity is preferably below 160 U/ml.

In addition, the present invention concerns a method for detecting the presence of or predisposition to autism, an autism spectrum disorder, or an autism-associated disorder in a subject, the method comprising detecting the presence of an alteration in the PON1 gene locus in a biological sample of said subject. The alteration can be one or several SNP(s). Preferably, said SNP is C-108T and/or Q192R. In particular, the presence of an allele T of SNP C-108T and/or of an allele R of SNP Q192R in a subject is indicative of the presence of or the predisposition to autism, an autism spectrum disorder, or an autism-associated disorder. Preferably, the biological sample is a blood sample, preferably a serum sample.

Preferably, the presence of an alteration in the PON1 gene locus is detected by sequencing, selective hybridization and/or selective amplification.

### BRIEF DESCRIPTION OF THE DRAWINGS

FIG. 1: Serum arylesterase activity by diagnostic status and PON1 genotypes (A=autistic (N=88), M=mother (N=38), F=father (N=37), S=unaffected sibling (N=55), C=controls (N=60)). *** p<0.001. C-108T SNP=CC, CT or TT. Q192R SNP=QQ, QR or RR.

FIG. 2: Serum arylesterase activity measured in 88 autistic patients vs 60 normal controls. Arylesterase activity (“Aryl tot”) is expressed in U/ml. The cut-off at 160 U/ml is highlighted by the hyphenated line. FIG. 2A: All patients and controls, regardless of PON1 genotype. FIG. 2B: Stratification by PON1 Q192R: QQ genotype. FIG. 2C: Stratification by PON1 Q192R: QR genotype. FIG. 2D: Stratification by PON1 Q192R: RR genotype. FIG. 2E: Stratification by PON1 C-108T: CC genotype. FIG. 2F: Stratification by PON1 C-108T: CT genotype. FIG. 2G: Stratification by PON1 C-108T: TT genotype. FIG. 2H: Stratification by PON1 haplotypes: R−/T−. FIG. 2I: Stratification by PON1 haplotypes: R+/T−. FIG. 2J: Stratification by PON1 haplotypes: R−/T+. FIG. 2K: Stratification by PON1 haplotypes: R+/T+.

FIG. 3: Serum arylesterase activity measured in 88 autistic patients (A), 130 first-degree relatives including 38 mothers (M), 37 fathers (F), and 55 unaffected brothers and sisters (S), and in 60 normal controls (C). Arylesterase activity (“Aryl tot”) is expressed in U/ml. The cut-off at 160 U/ml is highlighted by the hyphenated line.

Table 1: Distributions of serum arylesterase activity in ASD patients and controls with T1χ² statistics; relative risks and 95% confidence intervals for each class of arylesterase activity contrasted against the rest of the sample; epidemiological coefficients refer to the <160 U/L class; R+, R allele present (QR+RR genotypes); R−, R allele absent (QQ genotype); T+, T allele present (CT+TT genotypes), T−, T allele absent (CC genotype).

Table 2: Distributions of serum arylesterase activity in 126 first-degree relatives of ASD patients and 60 normal controls with T1χ² statistics; relative risks and 95% confidence intervals for each class of arylesterase activity contrasted against the rest of the sample; epidemiological coefficients refer to the <160 U/L class.

Table 3: Distributions of serum arylesterase activity in 83 ASD patients and 57 unaffected siblings with T1χ² statistics; relative risks and 95% confidence intervals for each class of arylesterase activity contrasted against the rest of the sample; epidemiological coefficients refer to the <160 U/L class.

### DETAILED DESCRIPTION OF THE INVENTION

### Definitions

Autism and autism spectrum disorders (ASDs): Autism is typically characterized as part of a spectrum of disorders (ASDs) including Asperger syndrome (AS) and other pervasive developmental disorders (PPD). Autism shall be construed as any condition of impaired social interaction and communication with restricted repetitive and stereotyped patterns of behavior, interests and activities present before the age of 3, to the extent that health may be impaired. AS is distinguished from autistic disorder by the lack of a clinically significant delay in language development in the presence of the impaired social interaction and restricted repetitive behaviors, interests, and activities that characterize the autism-spectrum disorders (ASDs). PPD-NOS (PPD, not otherwise specified) is used to categorize children who do not meet the strict criteria for autism but who come close, either by manifesting atypical autism or by nearly meeting the diagnostic criteria in two or three of the key areas.

Autism-associated disorders, diseases or pathologies include, more specifically, any metabolic and immune disorders, epilepsy, anxiety, depression, attention deficit hyperactivity disorder, speech delay or language impairment, motor incoordination, schizophrenia and bipolar disorder.

Within the context of this invention, the PON1 gene locus designates all PON1 sequences or products in a cell or organism, including PON1 coding sequences, PON1 non-coding sequences (e.g., introns), PON1 regulatory sequences controlling transcription, translation (e.g., promoter, enhancer, terminator, etc.), RNA and/or protein stability, as well as all corresponding expression products, such as PON1 RNAs (e.g., mRNAs) and PON1 polypeptides (e.g., a pre-protein and a mature protein). The PON1 gene locus also comprises surrounding sequences of the PON1 gene which include SNPs that are in linkage disequilibrium with SNPs located in the PON1 gene.

As used in the present application, the term “PON1 gene” designates the gene encoding the Paraoxonase 1, as well as variants, analogs and fragments thereof, including alleles thereof (e.g., germline mutations) which are related to susceptibility to autism and autism-associated disorders. The PON1 gene may also be referred to as esterase A, ESA and PON.

The term “gene” shall be construed to include any type of coding nucleic acid, including genomic DNA (gDNA), complementary DNA (cDNA), synthetic or semi-synthetic DNA, as well as any form of corresponding RNA. The term gene particularly includes recombinant nucleic acids encoding PON1, i.e., any non naturally occurring nucleic acid molecule created artificially, e.g., by assembling, cutting, ligating or amplifying sequences. A PON1 gene is typically double-stranded, although other forms may be contemplated, such as single-stranded. PON1 genes may be obtained from various sources and according to various techniques known in the art, such as by screening DNA libraries or by amplification from various natural sources. Recombinant nucleic acids may be prepared by conventional techniques, including chemical synthesis, genetic engineering, enzymatic techniques, or a combination thereof. Suitable PON1 gene sequences may be found on gene banks, such as Unigene Cluster for PON1 (Hs. 370995), GeneID 5444 and Unigene Representative Sequence NM_{—}000446.4.

A fragment of an PON1 gene designates any portion of at least about 8 consecutive nucleotides of a sequence as disclosed above, preferably at least about 15, more preferably at least about 20 nucleotides, further preferably of at least 30 nucleotides. Fragments include all possible nucleotide lengths between 8 and 100 nucleotides, preferably between 15 and 100, more preferably between 20 and 100.

PON1 polypeptide designates any protein or polypeptide encoded by an PON1 gene as disclosed above. The term “polypeptide” refers to any molecule comprising a stretch of amino acids. This term includes molecules of various lengths, such as peptides and proteins. The polypeptide may be modified, such as by glycosylations and/or acetylations and/or chemical reaction or coupling, and may contain one or several non-natural or synthetic amino acids. A specific example of PON1 polypeptide comprises all or part of the amino acid sequence NP_{—}000437.3.

The encoded Paraoxonase 1 (PON1) presents an arylesterase activity (EC: 3.1.1.2). The enzyme catalyzes the following reaction: phenyl acetate+H2O→phenol+acetate.

Typical stringent hybridisation conditions include temperatures above 30° C., preferably above 35° C., more preferably in excess of 42° C., and/or salinity of less than about 500 mM, preferably less than 200 mM. Hybridization conditions may be adjusted by the skilled person by modifying the temperature, salinity and/or the concentration of other reagents such as SDS, SSC, etc.

Within the context of the present invention, the term ‘diagnosis” includes the detection, monitoring, dosing, comparison, etc., at various stages, including early, pre-symptomatic stages, and late stages, in adults, children and pre-birth. Diagnosis typically includes the prognosis, the assessment of a predisposition or risk of development, the characterization of a subject to define most appropriate treatment (pharmacogenetics), etc.

The invention may be used in various subjects, particularly human, including adults, children and at the prenatal stage.

The present invention concerns a method for detecting the presence of or predisposition to autism, an autism spectrum disorder, or an autism-associated disorder in a subject, the method comprising measuring an arylesterase enzymatic activity in a biological sample of said subject, a low arylesterase enzymatic activity being indicative of the presence of or the predisposition to autism, an autism spectrum disorder, or an autism-associated disorder.

The biological sample can be a biological fluid such as blood, serum, plasma, saliva, or urine or a tissue sample. In a preferred embodiment, the biological sample is blood, serum or plasma. Optionally, said method comprises a previous step of providing a sample from a subject.

The arylesterase activity can be measured by any method known in the art, and for instance as disclosed in the experimental section. Kits for measuring arylesterase activity are commercially available (i.e., Arylesterase/paraoxonase assay kit: ZMC Catalog #: 0801199, ZeptoMetrix; Catalog #: 02-0801199, GENTAUR).

By “low” is intended that the arylesterase enzymatic activity is lower than the activity of a sample from a control subject. The control subject is a non-affected subject without any link to an affected subject. More precisely, the one skilled in the art can adapt the cut-off level of activity depending for instance to the used sample. In a preferred embodiment, for a serum sample, the cut-off activity is less than 200 U/ml, preferably less than 180 U/ml, and more preferably less than about 165 U/ml. An appropriate cut-off can be about 160 U/ml. By “about” is intended the value more or less than 5%. Accordingly, a low activity is less than 200 U/ml, preferably less than 180 U/ml, and more preferably less than about 165 U/ml, and still more preferably less than 160 U/ml.

Preferably, the method further comprises detecting the presence of an alteration in the PON1 gene locus in a biological sample of said subject. Alternatively, the present invention concerns a method for detecting the presence of or predisposition to autism, an autism spectrum disorder, or an autism-associated disorder in a subject, the method comprising detecting the presence of an alteration in the PON1 gene locus in a biological sample of said subject. Optionally, said method comprises a previous step of providing a sample from a subject.

The presence of said alteration is indicative of the presence or predisposition to autism, an autism spectrum disorder, or an autism-associated disorder. Preferably, the presence of an alteration in the PON1 gene locus in said sample is detected through the genotyping of a sample.

When the method combines the arylesterase activity assessment and the detection of PON1 gene locus alteration, the sample can be the same for both determination or can be different. In particular, the sample may be any biological sample derived from a subject, which contains nucleic acids or polypeptides. Examples of such samples include fluids, tissues, cell samples, organs, biopsies, etc. Most preferred samples are blood, plasma, saliva, urine, etc.

The alteration can be one or several SNP(s). Preferably, said SNP is C-108T and/or Q192R. In a preferred embodiment, the alteration is a combination of C-108T and Q192R. The inventors have shown that the presence of an allele T of SNP C-108T and/or of an allele R (G) of SNP Q192R in a subject is indicative of the presence of or the predisposition to autism, an autism spectrum disorder, or an autism-associated disorder.

| | | | | Nucleotide | | |
|---|---|---|---|---|---|---|
| | | | | position in | | |
| | | | | genomic | | |
| | | | | sequence of | | |
| | | | | chromosome 7 | Position | SEQ |
| SNP | dbSNP | Allele | Allele | based on | in | ID |
| identity | reference | 1 | 2 | NT_007933 | locus | NO: |
| C-108T | rs 705379 | C | T | 20188171 | 5′ end | 6 |
| Q192R | rs 662 | A | G | 20170722 | Exon 6 | 7 |

The alteration may be determined at the level of the PON1 gDNA, RNA or polypeptide. Optionally, the detection is performed by sequencing all or part of the PON1 gene or by selective hybridization or amplification of all or part of the PON1 gene. More preferably a PON1 gene specific amplification is carried out before the alteration identification step.

In any method according to the present invention, one or several SNP in the PON1 gene and certain haplotypes comprising SNP in the PON1 gene can be used in combination with other SNP or haplotype associated with autism and associated disorders and located in other gene(s).

An alteration in the PON1 gene locus may be any form of mutation(s), deletion(s), rearrangement(s) and/or insertions in the coding and/or non-coding region of the locus, alone or in various combination(s). Mutations more specifically include point mutations. Deletions may encompass any region of two or more residues in a coding or non-coding portion of the gene locus, such as from two residues up to the entire gene or locus. Typical deletions affect smaller regions, such as domains (introns) or repeated sequences or fragments of less than about 50 consecutive base pairs, although larger deletions may occur as well. Insertions may encompass the addition of one or several residues in a coding or non-coding portion of the gene locus. Insertions may typically comprise an addition of between 1 and 50 base pairs in the gene locus. Rearrangement includes inversion of sequences. The PON1 gene locus alteration may result in the creation of stop codons, frameshift mutations, amino acid substitutions, particular RNA splicing or processing, product instability, truncated polypeptide production, etc. The alteration may result in the production of a PON1 polypeptide with altered function, stability, targeting or structure. The alteration may also cause a reduction in protein expression or, alternatively, an increase in said production.

In a particular embodiment of the method according to the present invention, the alteration in the PON1 gene locus is selected from a point mutation, a deletion and an insertion in the PON1 gene or corresponding expression product, more preferably a point mutation and a deletion.

In another variant, the method comprises detecting the presence of an altered PON1 RNA expression. Altered RNA expression includes the presence of an altered RNA sequence, the presence of an altered RNA splicing or processing, the presence of an altered quantity of RNA, etc. These may be detected by various techniques known in the art, including by sequencing all or part of the PON1 RNA or by selective hybridization or selective amplification of all or part of said RNA, for instance.

In a further variant, the method comprises detecting the presence of an altered PON1 polypeptide expression. Altered PON1 polypeptide expression includes the presence of an altered polypeptide sequence, the presence of an altered quantity of PON1 polypeptide, the presence of an altered tissue distribution, etc. These may be detected by various techniques known in the art, including by sequencing and/or binding to specific ligands (such as antibodies), for instance.

As indicated above, various techniques known in the art may be used to detect or quantify altered PON1 gene or RNA expression or sequence, including sequencing, hybridization, amplification and/or binding to specific ligands (such as antibodies). Other suitable methods include allele-specific oligonucleotide (ASO), allele-specific amplification, Southern blot (for DNAs), Northern blot (for RNAs), single-stranded conformation analysis (SSCA), PFGE, fluorescent in situ hybridization (FISH), gel migration, clamped denaturing gel electrophoresis, heteroduplex analysis, RNase protection, chemical mismatch cleavage, ELISA, radio-immunoassays (RIA) and immuno-enzymatic assays (IEMA).

Some of these approaches (e.g., SSCA and CGGE) are based on a change in electrophoretic mobility of the nucleic acids, as a result of the presence of an altered sequence. According to these techniques, the altered sequence is visualized by a shift in mobility on gels. The fragments may then be sequenced to confirm the alteration.

Some others are based on specific hybridization between nucleic acids from the subject and a probe specific for wild type or altered PON1 gene or RNA. The probe may be in suspension or immobilized on a substrate. The probe is typically labeled to facilitate detection of hybrids.

Some of these approaches are particularly suited for assessing a polypeptide sequence or expression level, such as Northern blot, ELISA and RIA. These latter require the use of a ligand specific for the polypeptide, more preferably of a specific antibody.

In a particular, preferred, embodiment, the method comprises detecting the presence of an altered PON1 gene expression profile in a sample from the subject. As indicated above, this can be accomplished more preferably by sequencing, selective hybridization and/or selective amplification of nucleic acids present in said sample.

Sequencing

Sequencing can be carried out using techniques well known in the art, using automatic sequencers. The sequencing may be performed on the complete PON1 gene or, more preferably, on specific domains thereof, typically those known or suspected to carry deleterious mutations or other alterations.

Amplification

Amplification is based on the formation of specific hybrids between complementary nucleic acid sequences that serve to initiate nucleic acid reproduction.

Amplification may be performed according to various techniques known in the art, such as by polymerase chain reaction (PCR), ligase chain reaction (LCR), strand displacement amplification (SDA) and nucleic acid sequence based amplification (NASBA). These techniques can be performed using commercially available reagents and protocols. Preferred techniques use allele-specific PCR or PCR-SSCP. Amplification usually requires the use of specific nucleic acid primers, to initiate the reaction.

Nucleic acid primers useful for amplifying sequences from the PON1 gene or locus are able to specifically hybridize with a portion of the PON1 gene locus that flank a target region of said locus, said target region being altered in certain subjects having autism, an autism spectrum disorder, or an autism-associated disorder.

Primers that can be used to amplify PON1 target region comprising SNPs may be designed based on the mRNA or cDNA sequence or on the genomic sequence of PON1. In a particular embodiment, primers may be designed based on the sequence of SEQ ID Nos 1-5.

Typical primers of this invention are single-stranded nucleic acid molecules of about 5 to 60 nucleotides in length, more preferably of about 8 to about 25 nucleotides in length. The sequence can be derived directly from the sequence of the PON1 gene locus. Perfect complementarity is preferred, to ensure high specificity. However, certain mismatch may be tolerated.

The invention also concerns the use of a nucleic acid primer or a pair of nucleic acid primers as described above in a method of detecting the presence of or predisposition to autism, an autism spectrum disorder, or an autism-associated disorder in a subject.

Selective Hybridization

Hybridization detection methods are based on the formation of specific hybrids between complementary nucleic acid sequences that serve to detect nucleic acid sequence alteration(s).

A particular detection technique involves the use of a nucleic acid probe specific for wild type or altered PON1gene or RNA, followed by the detection of the presence of a hybrid. The probe may be in suspension or immobilized on a substrate or support (as in nucleic acid array or chips technologies). The probe is typically labeled to facilitate detection of hybrids.

In this regard, a particular embodiment of this invention comprises contacting the sample from the subject with a nucleic acid probe specific for an altered PON1gene locus, and assessing the formation of an hybrid. In a particular, preferred embodiment, the method comprises contacting simultaneously the sample with a set of probes that are specific, respectively, for wild type PON1gene locus and for various altered forms thereof. In this embodiment, it is possible to detect directly the presence of various forms of alterations in the PON1gene locus in the sample. Also, various samples from various subjects may be treated in parallel.

Within the context of this invention, a probe refers to a polynucleotide sequence which is complementary to and capable of specific hybridization with a (target portion of a) PON1 gene or RNA, and which is suitable for detecting polynucleotide polymorphisms associated with PON1 alleles which predispose to or are associated with autism, an autism spectrum disorder, or an autism-associated disorder. Probes are preferably perfectly complementary to the PON1 gene, RNA, or target portion thereof. Probes typically comprise single-stranded nucleic acids of between 8 to 1000 nucleotides in length, for instance of between 10 and 800, more preferably of between 15 and 700, typically of between 20 and 500. It should be understood that longer probes may be used as well. A preferred probe of this invention is a single stranded nucleic acid molecule of between 8 to 500 nucleotides in length, which can specifically hybridise to a region of a PON1 gene or RNA that carries an alteration.

A specific embodiment of this invention is a nucleic acid probe specific for an altered (e.g., a mutated) PON1 gene or RNA, i.e., a nucleic acid probe that specifically hybridises to said altered PON1 gene or RNA and essentially does not hybridise to a PON1 gene or RNA lacking said alteration. Specificity indicates that hybridization to the target sequence generates a specific signal which can be distinguished from the signal generated through non-specific hybridization. Perfectly complementary sequences are preferred to design probes according to this invention. It should be understood, however, that a certain degree of mismatch may be tolerated, as long as the specific signal may be distinguished from non-specific hybridization.

Particular examples of such probes are nucleic acid sequences complementary to a target portion of the genomic region including the PON1 gene or RNA carrying a point mutation of SNP C-108T or Q192R as defined above. More particularly, the probes can comprise a sequence selected from the group consisting of SEQ ID Nos 6-7 or a fragment thereof comprising the SNP or a complementary sequence thereof.

The sequence of the probes can be derived from the sequences of the PON1 gene and RNA as provided in the present application. Nucleotide substitutions may be performed, as well as chemical modifications of the probe. Such chemical modifications may be accomplished to increase the stability of hybrids (e.g., intercalating groups) or to label the probe. Typical examples of labels include, without limitation, radioactivity, fluorescence, luminescence, enzymatic labeling, etc.

The invention also concerns the use of a nucleic acid probe as described above in a method of detecting the presence of or predisposition to autism, an autism spectrum disorder, or an autism-associated disorder in a subject.

### Specific Ligand Binding

As indicated above, alteration in the PON1 gene locus may also be detected by screening for alteration(s) in PON1 polypeptide sequence or expression levels (e.g., the presence of an amino acid R or Q at position 192 of PON1 polypeptide). In this regard, a specific embodiment of this invention comprises contacting the sample with a ligand specific for a PON1 polypeptide and determining the formation of a complex.

Different types of ligands may be used, such as specific antibodies. In a specific embodiment, the sample is contacted with an antibody specific for a PON1 polypeptide and the formation of an immune complex is determined. Various methods for detecting an immune complex can be used, such as ELISA, radioimmunoassays (RIA) and immuno-enzymatic assays (IEMA).

Within the context of this invention, an antibody designates a polyclonal antibody, a monoclonal antibody, as well as fragments or derivatives thereof having substantially the same antigen specificity. Fragments include Fab, Fab'2, CDR regions, etc. Derivatives include single-chain antibodies, humanized antibodies, poly-functional antibodies, etc.

An antibody specific for a PON1 polypeptide designates an antibody that selectively binds a PON1 polypeptide, namely, an antibody raised against a PON1 polypeptide or an epitope-containing fragment thereof. Although non-specific binding towards other antigens may occur, binding to the target PON1 polypeptide occurs with a higher affinity and can be reliably discriminated from non-specific binding.

In a specific embodiment, the method comprises contacting a sample from the subject with (a support coated with) an antibody specific for an altered form of a PON1 polypeptide, and determining the presence of an immune complex. In a particular embodiment, the sample may be contacted simultaneously, or in parallel, or sequentially, with various (supports coated with) antibodies specific for different forms of a PON1 polypeptide, such as a wild type and various altered forms thereof.

The invention also concerns the use of a ligand, preferably an antibody, a fragment or a derivative thereof as described above, in a method of detecting the presence of or predisposition to autism, an autism spectrum disorder, or an autism-associated disorder in a subject.

In order to carry out the methods of the invention, one can employ diagnostic kits comprising products and reagents for detecting in a sample from a subject the presence of an alteration in the PON1 gene or polypeptide, in the PON1 gene or polypeptide expression, and/or in PON1 activity. Said diagnostic kit comprises any primer, any pair of primers, any nucleic acid probe and/or any ligand, preferably antibody, described in the present invention. Said diagnostic kit can further comprise reagents and/or protocols for performing a hybridization, amplification, antigen-antibody immune reaction and/or arylesterase activity assay.

The sample may be collected according to conventional techniques and used directly for diagnosis or stored. The sample may be treated prior to performing the method, in order to render or improve availability of nucleic acids or polypeptides for testing. Treatments include, for instant, lysis (e.g., mechanical, physical, chemical, etc.), centrifugation, etc. Also, the nucleic acids and/or polypeptides may be pre-purified or enriched by conventional techniques, and/or reduced in complexity. Nucleic acids and polypeptides may also be treated with enzymes or other chemical or physical treatments to produce fragments thereof. Considering the high sensitivity of the claimed methods, very few amounts of sample are sufficient to perform the assay.

As indicated, the sample is preferably contacted with reagents such as probes, primers or ligands in order to assess the presence of an altered PON1 gene locus. Contacting may be performed in any suitable device, such as a plate, tube, well, glass, etc. In specific embodiments, the contacting is performed on a substrate coated with the reagent, such as a nucleic acid array or a specific ligand array. The substrate may be a solid or semi-solid substrate such as any support comprising glass, plastic, nylon, paper, metal, polymers and the like. The substrate may be of various forms and sizes, such as a slide, a membrane, a bead, a column, a gel, etc. The contacting may be made under any condition suitable for a complex to be formed between the reagent and the nucleic acids or polypeptides of the sample.

The finding of an altered PON1 polypeptide, RNA or DNA in the sample is indicative of the presence of an altered PON1 gene locus in the subject, which can be correlated to the presence, predisposition or stage of progression of autism, an autism spectrum disorder, or an autism-associated disorder. For example, an individual having a germ line PON1 mutation has an increased risk of developing autism, an autism spectrum disorder, or an autism-associated disorder. The determination of the presence of an altered PON1 gene locus in a subject also allows the design of appropriate therapeutic intervention, which is more effective and customized. Also, this determination at the pre-symptomatic level allows a preventive regimen to be applied.

### Linkage Disequilibirum

Once a first SNP has been identified in a genomic region of interest, more particularly in PON1 gene locus, the practitioner of ordinary skill in the art can easily identify additional SNPs in linkage disequilibrium with this first SNP. Indeed, any SNP in linkage disequilibrium with a first SNP associated with autism, an autism spectrum disorder, or an autism-associated disorder will be associated with this trait. Therefore, once the association has been demonstrated between a given SNP and autism, an autism spectrum disorder, or an autism-associated disorder, the discovery of additional SNPs associated with this trait can be of great interest in order to increase the density of SNPs in this particular region.

Identification of additional SNPs in linkage disequilibrium with a given SNP involves: (a) amplifying a fragment from the genomic region comprising or surrounding a first SNP from a plurality of individuals; (b) identifying of second SNPs in the genomic region harboring or surrounding said first SNP; (c) conducting a linkage disequilibrium analysis between said first SNP and second SNPs; and (d) selecting said second SNPs as being in linkage disequilibrium with said first marker. Subcombinations comprising steps (b) and (c) are also contemplated.

Methods to identify SNPs and to conduct linkage disequilibrium analysis can be carried out by the skilled person without undue experimentation by using well-known methods.

These SNPs in linkage disequilibrium can also be used in the methods according to the present invention, and more particularly in the diagnosic methods according to the present invention.

### Causal Mutation

Mutations in the PON1 gene which are responsible for autism or an associated disorder may be identified by comparing the sequences of the PON1 gene from patients presenting autism or an associated disorder and control individuals. Based on the identified association of SNPs of PON1 and autism or an associated disorder, the identified locus can be scanned for mutations. In a preferred embodiment, functional regions such as exons and splice sites, promoters and other regulatory regions of the PON1 gene are scanned for mutations. Preferably, patients presenting autism or an associated disorder carry the mutation shown to be associated with autism or an associated disorder and controls individuals do not carry the mutation or allele associated with autism or an associated disorder. It might also be possible that patients presenting autism or an associated disorder carry the mutation shown to be associated with autism or an associated disorder with a higher frequency than controls individuals.

The method used to detect such mutations generally comprises the following steps: amplification of a region of the PON1 gene comprising a SNP or a group of SNPs associated with autism or an associated disorder from DNA samples of the PON1 gene from patients presenting autism or an associated disorder and control individuals; sequencing of the amplified region; comparison of DNA sequences of the PON1 gene from patients presenting autism or an associated disorder and control individuals; determination of mutations specific to patients presenting autism or an associated disorder.

Therefore, identification of a causal mutation in the PON1 gene can be carried out by the skilled person without undue experimentation by using well-known methods.

The present invention demonstrates the correlation between autism or an associated disorder and the PON1 gene locus. The invention thus provides a novel target of therapeutic intervention. Various approaches can be contemplated to restore or modulate the PON1 activity or function in a subject, particularly those carrying an altered PON1 gene locus. Supplying wild-type function to such subjects is expected to suppress phenotypic expression of autism or an associated disorder in a pathological cell or organism. The supply of such function can be accomplished through gene or protein therapy, or by administering compounds that modulate or mimic PON1 polypeptide activity (e.g., agonists as identified in the above screening assays).

Restoration of functional PON1 gene function in a cell may be used to prevent the development of autism or an associated disorder or to reduce progression of said diseases. Such a treatment may suppress autism-associated phenotype of a cell, particularly those cells carrying a deleterious allele.

Further aspects and advantages of the present invention will be disclosed in the following experimental section, which should be regarded as illustrative and not limiting the scope of the present application.

### EXAMPLES

### Materials and Methods

A) Measurement of serum arylesterase enzymatic activity

Arylesterase enzymatic activity was measured in plasma using standard spectrophotometric procedures, hereby described in detail:
1) The following solutions were prepared: (a) assay buffer (9.0 mM Tris-HCl, pH 8.0, +0.9 mM CaCl₂); (b) substrate solution (3.26 mM phenylacetate in assay buffer). Since substrate solution is light- and temperature-sensitive, it was screened from light, kept on ice, and prepared fresh once every hour vortexing vigorously;
2) Each human serum sample was thawed, mixed briefly by pipetting, and 1 μl of serum was diluted 1:50 in doubly-distilled water; 1 μl of this 1:50 dilution was then added to 220 μl of assay buffer;
3) Substrate solution was vortexed again and 280 μl of substrate solution were pipetted into the quartz cuvette inserted into the spectrophotometer; 220 μl of serum diluted in assay buffer were then immediately added into the cuvette, to reach a final volume of 500 ml.
4) The enzymatic reaction was monitored by spectrophotometry using a SAFAS Monaco UV mc2 apparatus, measuring AOD min at 270 nm for two minutes at room temperature. Enzymatic activity was then quantified in Units/Liter as follows: U/L=(mean AOD min/1.31)*25,000 (where 1.31 is the molar extinction coefficient for phenol and 25,000 is the dilution factor).
5) Two independent dilutions and spectrophotometric measurements were performed for each individual serum sample. A third measurement was performed if the two initial measurements yielded differences greater than 20%. Each measure of arylesterase enzymatic activity is thus the mean of 2-to-3 independent measurements of the same serum sample. Validation was implemented by frequent blanking (negative control) and measuring the same test sample at least three times a day (positive control). Measures were not considered valid and were repeated in the presence of initial ABS ≧0.8 (usually ABS ranges between 0.2 and 0.4).

B) Assessment of the Q192R and C-108T Single Nucleotide Polymorphisms

The PON1 C-108T and Q192R single nucleotide polymorphisms (SNPs) were each genotyped separately by PCR amplification and restriction digest, as described. Briefly: (a) the C-108T SNP was amplified using primer F: GACCGCAAGCCACGCCTTCTGTGCACC (SEQ ID No 1) and primer R: TGCAGCCGCAGCCCTGCTGGGGCAGCGCCGATTGGCCCGCCGC (SEQ ID No 2) with 5% DMSO and an annealing temperature of 63° C. for 35 cycles. The 109 by fragment was digested with BstUI, yielding 67 and 42 by fragments in the presence of the C allele; (b) the Q192R SNP was amplified using primer F: TATTGTTGCTGTGGGACCTGAG (SEQ ID No 3) and primer R: CACGCTAAACCCAAATACATCTC (SEQ ID No 4) at an annealing temperature of 60° C. for 35 cycles. The 99 by fragment was digested with AlwI, yielding 66 and 33 by fragments with the R allele.

In addition to using this classical method, the Q192R SNP was genotyped also by Template-directed Dye-terminator Incorporation with Fluorescence Polarization (TDI-FP), using the same primers shown above for PCR amplification, and the following SNP primer for dye-terminator incorporation: TGATCACTATTTTCTTGACCCCTACTTAC (SEQ ID No 5). PCR reactions were set up using MULTIPLATE 96 well plates (MJ Research, Cat. MLP-9601), as follows:

| | |
|---|---|
| Water | 3.55 μl |
| 10x buffer | 0.62 μl |
| dNTPs (2.5 mM each) | 0.25 μl |
| Primer F (20 pM) | 0.25 μl |
| Primer R (20 pM) | 0.25 μl |
| Taq polimerase | 0.08 μl |
| DNA (50 ng) | 1.25 μl of a 40 ng/μl dilution |
| Total Volume | 6.25 μl |

PCR reactions were then run at 95° C. for 3 min, followed by 35 cycles at 95° C. for 1 min, 60° C. for 1 min and 72° C. for 1 min, followed by 72° C. for 8 min. PCR clean-up was performed in black Hard-shell Thin-Wall microplates (MJ Research), modifying the manufacturer protocol (Perkin-Elmer) with the addition of exonuclease I (New England Biolabs, code #MO2935) and alkaline phosphatase (Sigma-Aldrich, code #P-9088), as follows:

| | | |
|---|---|---|
| Water | 3.63 | μl |
| PCR Clean-up dilution buffer | 1.65 | μl |
| Exonuclease | 0.04 | μl |
| Alkaline phosphatase | 0.33 | μl |
| PCR Clean-up reagent 10X | 0.2 | μl |
| PPase reagent | 0.15 | μl |
| PCR reaction | 1 | μl |
| Total volume | 7 | μl |

Clean-up reactions were performed at 37° C. for 60 min, followed by enzyme inactivation at 80° C. for 15 min. Finally, primer extension with the fluorescent dye-terminator was performed preparing the AcycloPrime-FP Mix according to the manufacturer's instructions, as follows:

| | | |
|---|---|---|
| Water | 9.45 | μl |
| 10X Reaction buffer | 2 | μl |
| AcycloTerminator Mix | 1 | μl |
| SNP primer (10 μM) | 0.5 | μl |
| AcycloPol | 0.05 | μl |
| Total volume | 13 | μl |

A total of 13 μl of reaction mix were diluted to a final volume of 20 μl, placed on a thermal cycler and run at 95° C. for 2 min, followed by a total of 20, 35, and 50 cycles at 95° C. for 15 sec, followed by 55° C. for 30 sec. After 20, 35, and 50 cycles, fluorescent polarization from R110 and TAMRA was read for 0.2 sec using a Victor2 plate reader, yielding QQ, QR, and RR genotypes. Negative and positive controls were included in each plate to ensure genotyping reliability.

Results

1) The inventors measured serum arylesterase activity in 278 individuals, including 88 patients with non-syndromic autism-spectrum disorder, 130 first-degree relatives belonging to 3 simplex (i.e., with 1 autistic child) and 36 multiplex (i.e. with two or more autistic children) families, and 60 normal controls (FIG. 1):

(a) On average, arylesterase activity is dramatically decreased in autistic patients compared to normal controls (P<0.001), regardless of PON1 genotypes.

Affection status effect: *** P<0.001 * P<0.05

The inventors also recorded a statistically significant difference between autistic patients and their first-degree relatives (P<0.05), though less pronounced compared to the difference between patients and normal controls;

(b) The C-108T SNP significantly influences arylesterase activity both in affected and unaffected individuals. The T-108 allele is associated with lower mean enzymatic activities;

T-108 allelic effect: Controls, TT<CT=CC (P<0.01)
A+M+F+S, TT=CT<CC (P<0.01)

(c) The Q192R SNP significantly influences arylesterase activity, but the R192 allele is associated with lower mean enzymatic activities only in autistic patients and their first-degree relatives, not in normal individuals.

Autism-specific R192 effect: A+M+F+S: RR=QR<QQ (P<0.01)

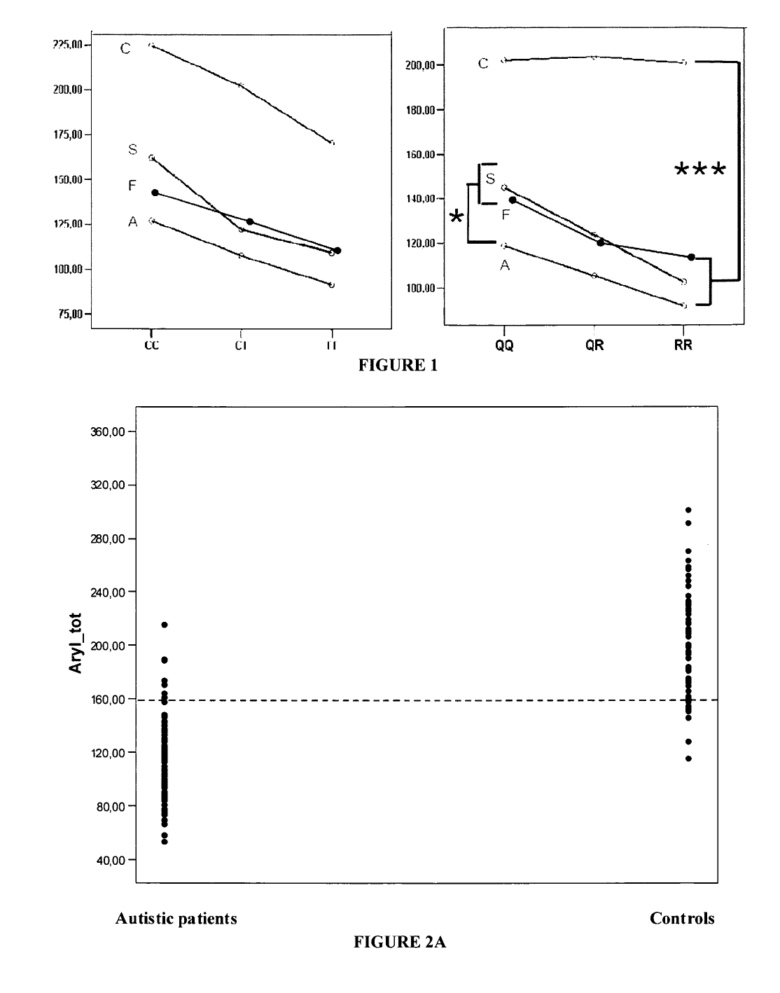

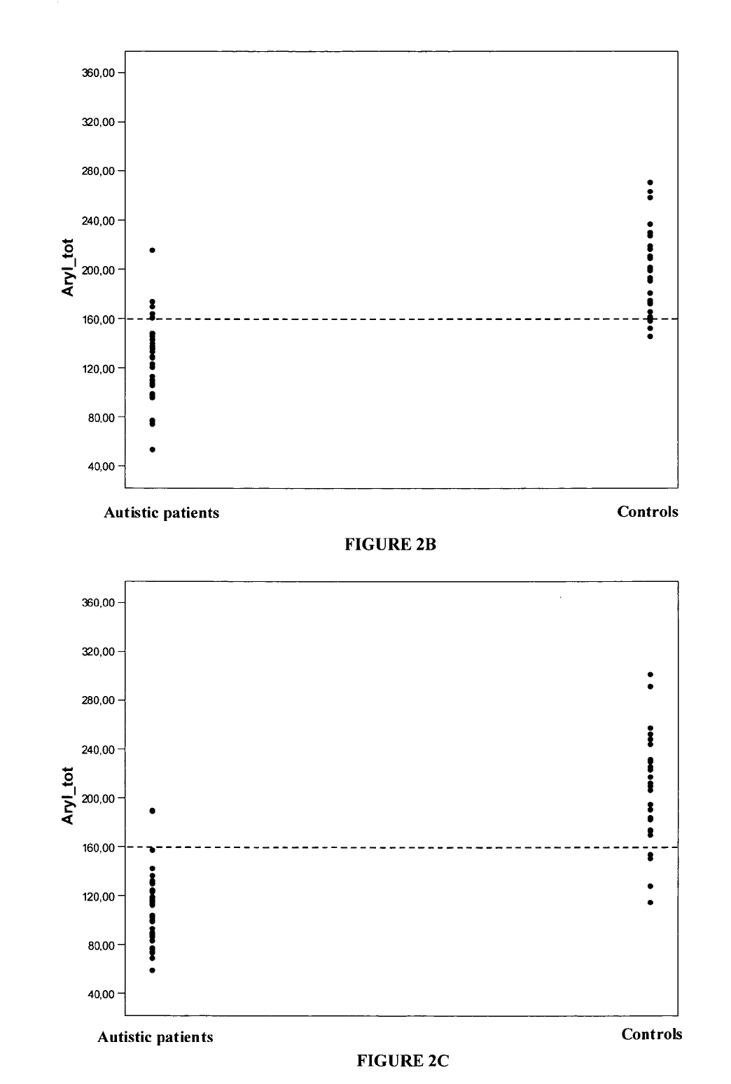

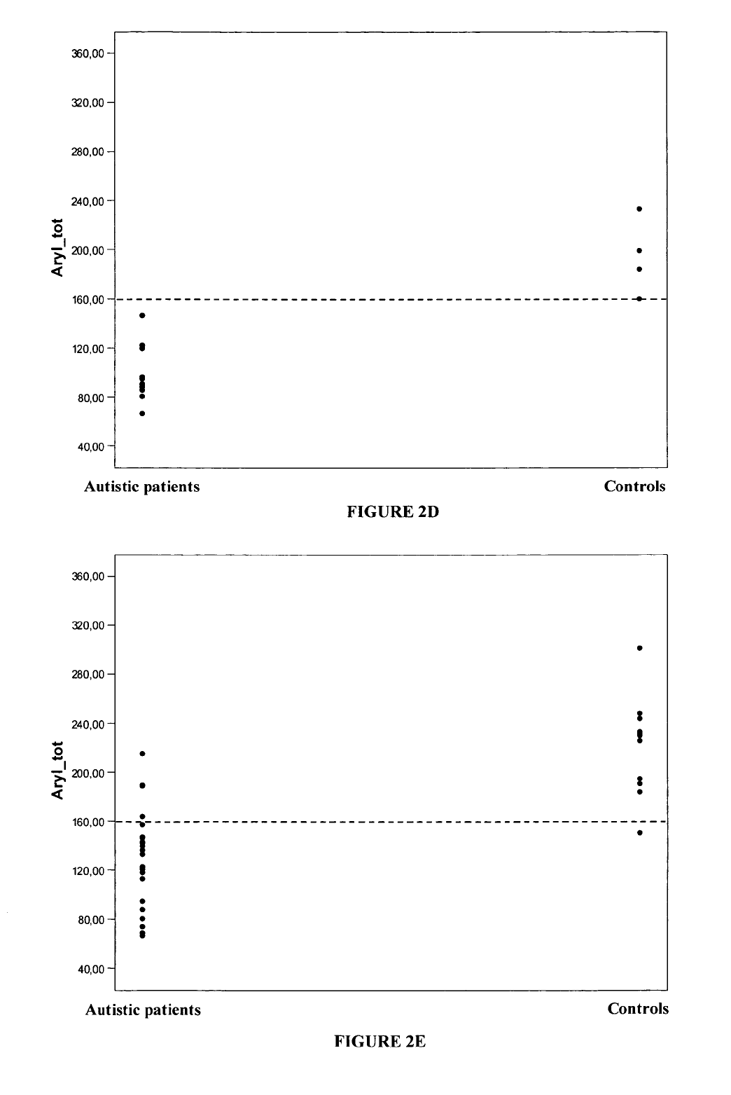

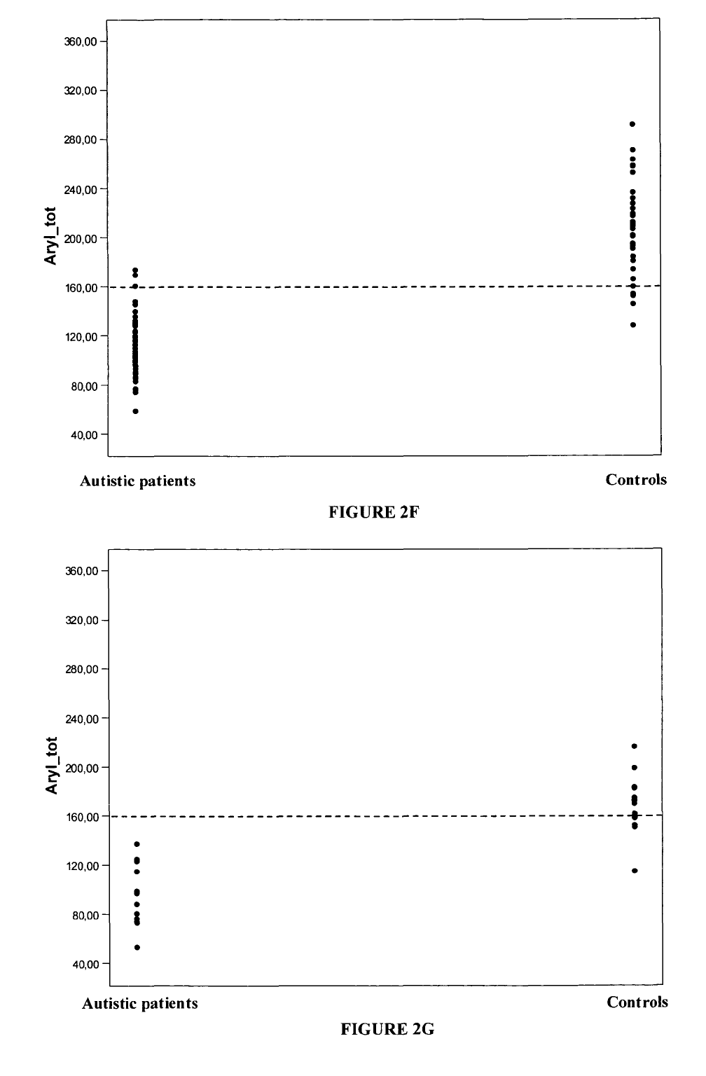

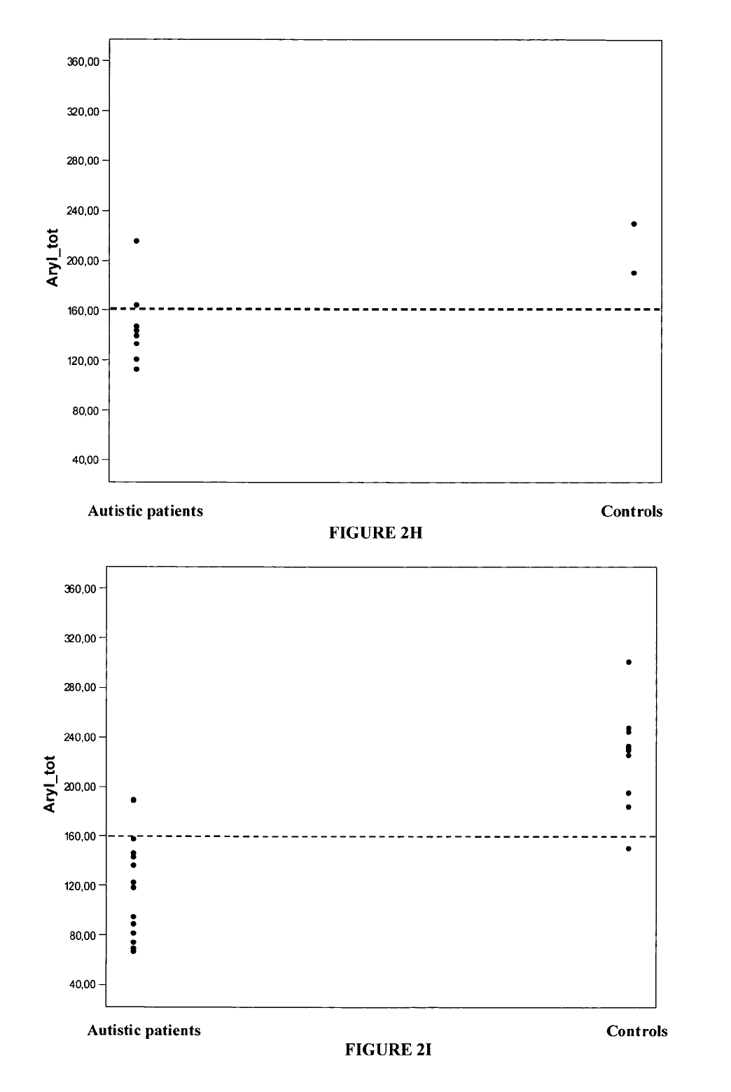

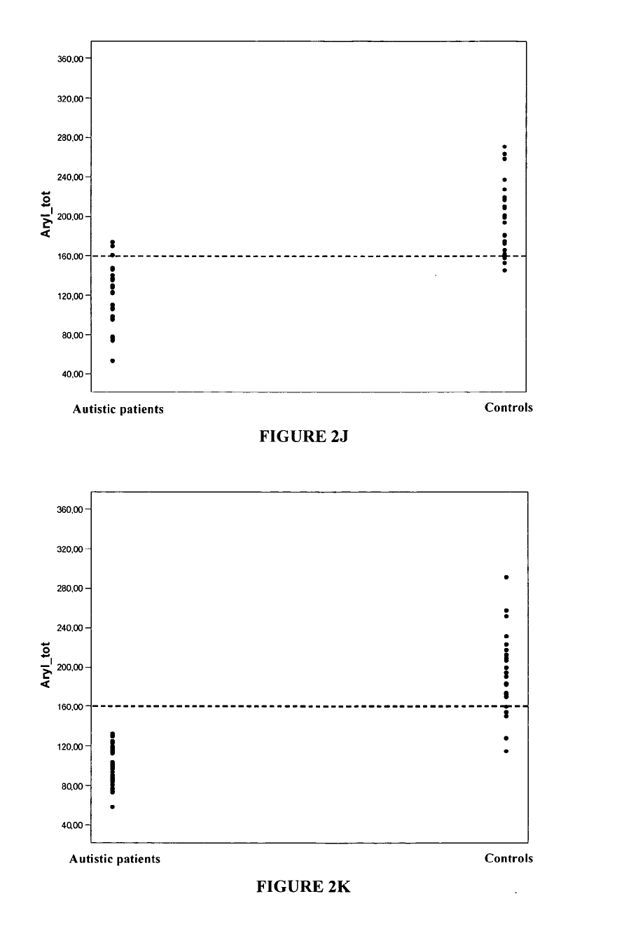

2) These data were then analyzed by quartilic classes, in order to calculate the sensitivity, specificity, positive and negative predictive values of serum arylesterase activity used in autism diagnostics, either alone or in combination with the PON1 C-108T, Q192R, or both SNPs. In order to interpret these results, it has to be reminded that (1) quartilic distributions are determined on the basis of control data, and then the distribution of patients is contrasted against the distribution in controls. Quartilic classes based on the distribution of serum arylesterase activity recorded in our patients were as follows: <160 U/ml (percentile 0-24), 160-199.9 U/ml (percentile 25-49), 200-239.9 U/ml (percentile 50-74), >240 U/ml (percentile 75-100). According to the present data, <160 U/ml represents the most reliable cut-off to distinguish affected and unaffected individuals; (2) “relative risk” for each quartilic class of arylesterase activity is the ratio between the percentage of autistic patients within that quartilic class, over the percentage of autistic patients present into all other quartilic classes combined; (3) “sensitivity” is the percentage of affected individuals identified as “affected” by our test, among all affected individuals; “specificity” is the percentage of unaffected individuals identified as “not affected” by our test, among all unaffected individuals; “positive predictive value” is the percentage of truly affected individuals, among all those individuals resulting positive at our test; “negative predictive value” is the percentage of truly unaffected individuals, among all those resulting negative at our test. Results summarized in parallel both in Table 1 and FIG. 2, provide clear evidence of the potential application of arylesterase activity (alone, but even better in combination with both SNPs), as a diagnostic tool for autism-spectrum disorders. In particular, belonging to the 0-25 quartilic class (i.e., serum arylesterase activity levels below 160 U/ml, as highlighted in FIG. 2 by the hyphenated line) is associated with a 7.41 relative risk of receiving a diagnosis of autism or of an autism-spectrum disorder. Carrying one or two copies of the R192 and T-108 alleles, increases these figures to impressive 13.61 and 12.57 relative risks, whereas carrying the QQ or the CC genotypes slightly reduces relative risks, which nonetheless remain at 4.88 and 3.56, respectively (Table 1 and FIG. 2).

The power of arylesterase activity as a diagnostic marker, in combination with the Q192R and C-108T SNPs, is also evidenced by the sensitivity, specificity, positive and negative predictive values summarized in Table 1. In simple terms, using a cut-off at <160 U/ml, there only is 8.4% of all autistic individuals with higher enzymatic activity (sensitivity=91.6%), and 15.0% of all non-autistic individuals with lower enzymatic activity (specificity=85.0%). If a blood test is performed and serum arylesterase activity is measured blind to patient condition (as would occur in a normal clinical setting), there is an 89.4% probability of correctly classifying as “affected” an individual who is indeed autistic (positive predictive value), and an 87.9% probability of classifying as “not affected” an individual who is not autistic (negative predictive value). These positive and negative predictive values are to a variable extent dependent upon Q192R and C-108T genotypes and haplotypes. The importance of using both Q192R and C-108T genotypes is exemplified by analyses focussed on the R+/T+haplotype (Table 1, last set). If an individual carries at least one copy of the R192 allele (i.e., his/her PON1 Q192R genotype is either QR or RR), and at least one copy of the T-108 allele (i.e., his/her PON1 C-108T genotype is either CT or TT), an arylesterase activity equal or greater than 160 U/ml gives the absolute certainty, at this stage in our studies, that the individual will not suffer from autism-spectrum disorders. If instead arylesterase activity is below 160 U/ml, there is a 90% probability that this individual is, or will be diagnosed with autism-spectrum disorder.

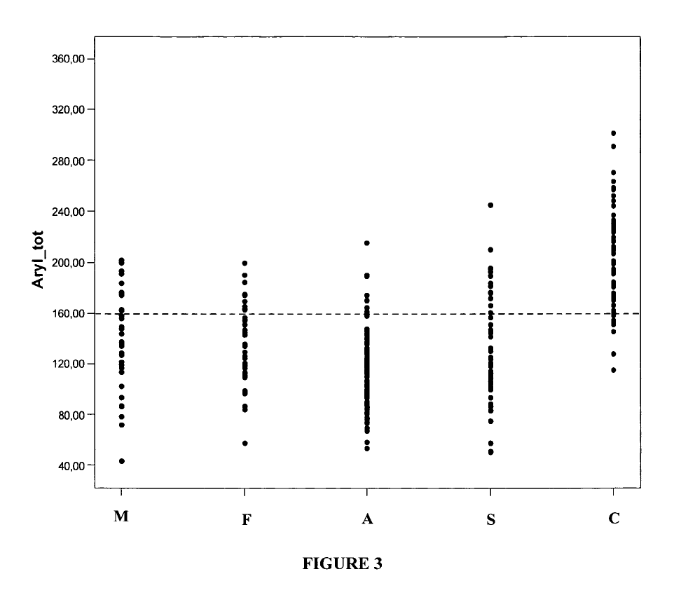

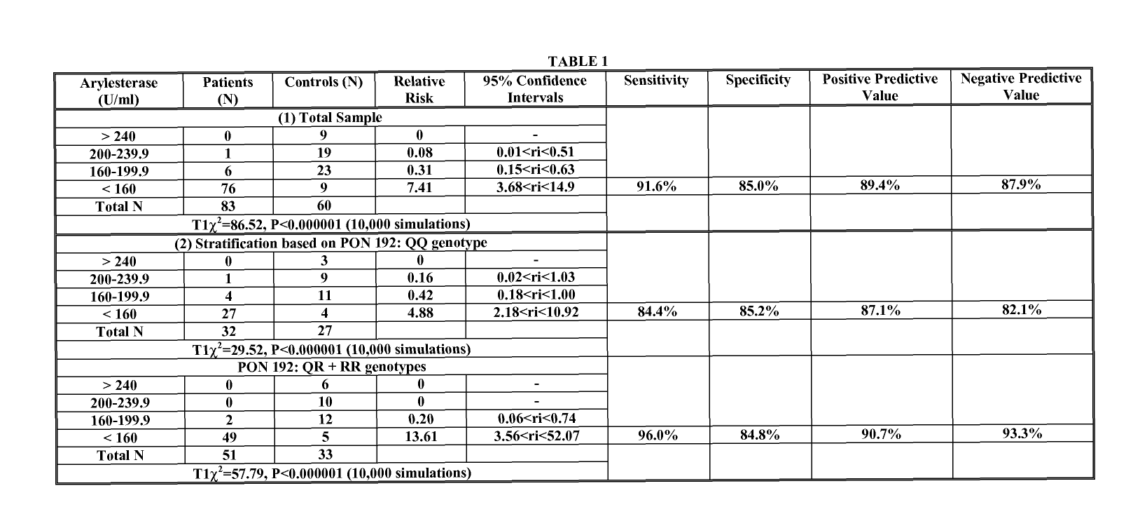

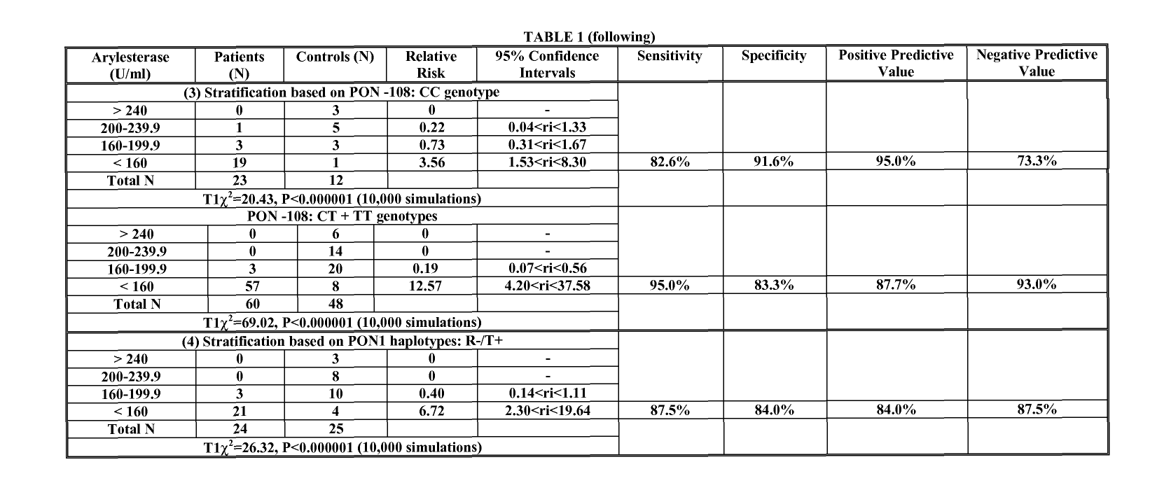

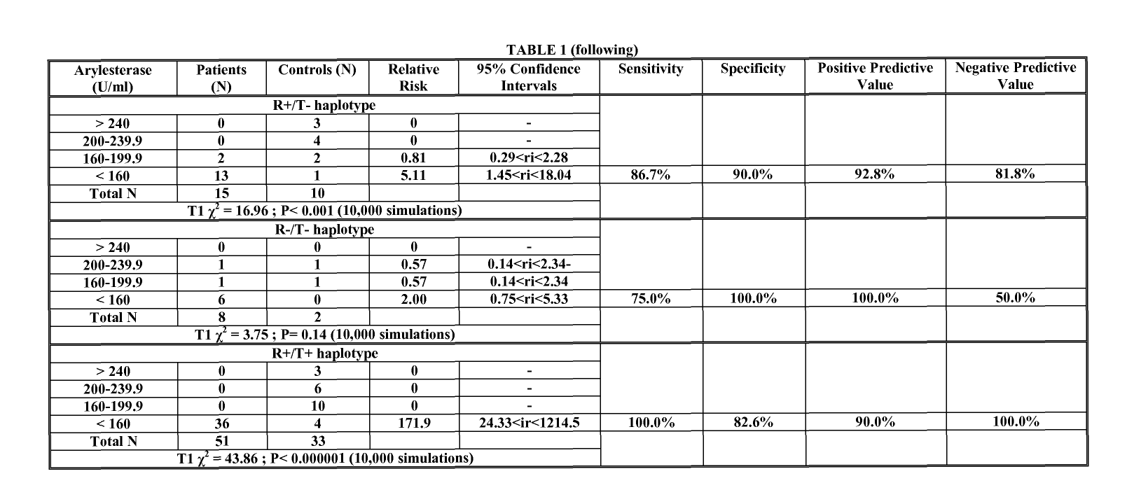

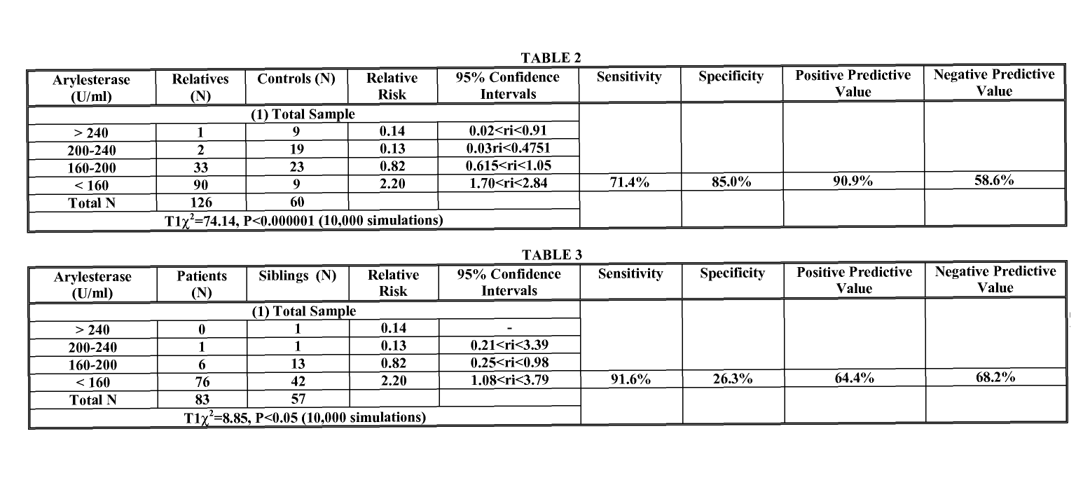

3) The inventors have finally calculated risk parameters for autistic patients, compared to their non-autistic first-degree relatives, including mothers, fathers, and unaffected brothers and sisters. Arylesterase activity suggests that first-degree relatives of autistic patients may represent “unaffected carriers” of the same pathological process affecting their autistic relatives: indeed, their serum arylesterase activity is much closer to activity levels recorded in autistic patients, than among controls (FIGS. 1 and 3).

In principle, this could occur because first-degree relatives either carry a protective genetic predisposition that limits the underlying pathophysiological damage, or they could carry a slightly lower genetic vulnerability load.

The present patent application seeks to protect:
1) All uses of arylesterase activity, measured in serum or in any other biological fluid or tissue by whatsoever technical method, as a diagnostic marker for autism-spectrum disorders (including autistic disorder, childhood disintegrative disorder, pervasive development disorder not otherwise specified and Asperger syndrome).
2) All uses of single nucleotide polymorphisms located in the PON1 gene, genotyped by whatsoever technical method, as a diagnostic marker for autism-spectrum disorders, including autistic disorder, childhood disintegrative disorder, pervasive development disorder not otherwise specified (PDD-NOS or “atypical autism”) and Asperger syndrome. All SNPs in the PON1 gene locus are included in this patent, and not only the Q192R and C-108T SNPs, because SNPs distributed throughout the human genome cosegregate non-independently (i.e., they are inherited in clusters, called “linkage disequilibrium blocks”); therefore, all other SNPs located in this gene would indirectly reflect the influence exerted by Q192R and C-108T;
3) All uses of arylesterase activity and/or PON1 SNPs C-108T and Q192R, aimed at identifying in the general population normal individuals carrying autism vulnerability and therefore at risk of having children with autism-spectrum disorders;
4) All uses of arylesterase activity and/or PON1 SNPs C-108T and Q192R, alone or in combination with any other test, aimed at identifying, either prenatally or postnatally, individuals suffering from autism-spectrum disorder, in families where at least one child has already been diagnosed with an autism-spectrum disorder.

1. A method for detecting the presence of or predisposition to autism or an autism spectrum disorder in a subject, the method comprising measuring an arylesterase enzymatic activity in a biological sample of said subject, a low arylesterase enzymatic activity being indicative of the presence of or the predisposition to autism, an autism spectrum disorder, or an autism-associated disorder.

2. The method according to claim 1, wherein the method further comprises detecting the presence of an alteration in the PON1 gene locus in a biological sample of said subject.

3. The method according to claim 2, wherein the alteration is one or several SNP(s).

4. The method according to claim 3, wherein said SNP is C-108T and/or Q192R.

5. The method according to claim 4, wherein the presence of an allele T of SNP C-108T in a subject is indicative of the presence of or the predisposition to autismor an autism spectrum disorder, or an autism associated disorder.

6. The method according to claim 4, wherein the presence of an allele R of SNP Q192R in a subject is indicative of the presence of or the predisposition to autismor an autism spectrum disorder.

7. The method according claim 1, wherein the biological sample is a blood sample, preferably a serum sample.

8. The method according to claim 7, wherein a low arylesterase enzymatic activity is below 160 U/ml.

9-14. (canceled)

15. The method according to claim 2, wherein the presence of an alteration in the PON1 gene locus is detected by sequencing, selective hybridization and/or selective amplification.

16. The method according to claim 2, wherein the biological sample is a blood sample, preferably a serum sample.

17. The method according to claim 3, wherein the biological sample is a blood sample, preferably a serum sample.

18. The method according to claim 4, wherein the biological sample is a blood sample, preferably a serum sample.

19. The method according to claim 5, wherein the biological sample is a blood sample, preferably a serum sample.

20. The method according to claim 6, wherein the biological sample is a blood sample, preferably a serum sample.

21. The method according to claim 3, wherein the presence of an alteration in the PON1 gene locus is detected by sequencing, selective hybridization and/or selective amplification.

22. The method according to claim 4, wherein the presence of an alteration in the PON1 gene locus is detected by sequencing, selective hybridization and/or selective amplification.

23. The method according to claim 5, wherein the presence of an alteration in the PON1 gene locus is detected by sequencing, selective hybridization and/or selective amplification.

24. The method according to claim 6, wherein the presence of an alteration in the PON1 gene locus is detected by sequencing, selective hybridization and/or selective amplification.


SEQUENCE LISTING (7 sequences)

SEQ ID NO: 1 - DNA, 27 bp, artificial sequence
  primer
gaccgcaagccacgccttctgtgcacc

SEQ ID NO: 2 - DNA, 43 bp, artificial sequence
  primer
tgcagccgcagccctgctggggcagcgccgattggcccgccgc

SEQ ID NO: 3 - DNA, 22 bp, artificial sequence
  primer
tattgttgctgtgggacctgag

SEQ ID NO: 4 - DNA, 23 bp, artificial sequence
  primer
cacgctaaacccaaatacatctc

SEQ ID NO: 5 - DNA, 29 bp, artificial sequence
  primer
tgatcactattttcttgacccctacttac

SEQ ID NO: 6 - DNA, 511 bp, homo sapiens
  misc_feature (256)..(256): C-108T Y=C or T
aggaccgggatggcacaaagtgagtgctcaccaaagcttgactgtcctttcccatggcaatttacttcagcttgtttgatttcccctccccgactggactaggcacctattctctgtcttctctctttacagttggaaggagcaaaatgggacttttggctgaaagtgctgagctcctgcggtgggggctgaccgcaagccgcgccttctgtgcacctggtcggcccagctagctgcggacccggcggggaggggyggggcgggccaatcggcgctgccccagcagggctgcggctgcaggcaggcagagcctcctagcccgtcggtgtctgcgcccatcgatccctttgtctatccccgaccatggcgaagctgattgcgctcaccctcttggggatgggactggcactcttcaggaaccaccagtcttcttaccagtaagttgggttgtggtgagggctgcagccttcgtcctcctcactccctgccagctccacgagcccccagaaag

SEQ ID NO: 7 - DNA, 601 bp, homo sapiens
  misc_feature (301)..(301): Q192R R=A or G
tacagtgctataatcacctcctcagatttcttagaccaatgattgtctttaaggattgtatcggcaggactaatttcatattaattgtgttccattatagctagcacgaaggctccatcccatatcttgattttaggaatagacagtgaggaatgccagttaataatcctgtaatgttcaataccttcaccttatatattatgtgtgtatgttttaattgcagtttgaatgatattgttgctgtgggacctgagcacttttatggcacaaatgatcactattttcttgacccctacttacratcctgggagatgtatttgggtttagcgtggtcgtatgttgtctactatagtccaagtgaagttcgagtggtggcagaaggatttgattttgctaatggaatcaacatttcacccgatggcaagtatgtgaactctctgaaatgtagtggatttactcagattctcaggagatatcttggaatatattctttttttaagtaatatgatacattttaacatgttaccctagtgagataaaattaggaaaaatgtaaaatgtcttaatttttcagggggtgaaatttatcaaatcaaaaatt